(19) United States
(12) Patent Application Publication
(10) Pub. No.: US 2006/0217750 A1
(43) Pub. Date: Sep. 28, 2006

(54) Surgical knife

(75) Inventor: Ziad R. Ghannoum, Trabuco Canyon, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/090,555

(22) Filed: Mar. 25, 2005

(51) Int. Cl. A61B 17/32

(52) U.S. Cl. 606/167

(57) ABSTRACT

A surgical knife containing a stain or dye coating. The stain or dye will dissolve or melt upon entry into the incision, thereby neatly marking the location of the incision.

### BACKGROUND OF THE INVENTION

This invention relates generally to surgical knives and particularly to knives used in ophthalmic surgery.

For many years, the predominant method of treating a diseased lens has been to remove the diseased lens and replace it with an intraocular lens (“IOL”). Two surgical procedures are preferred for removing the diseased lens: extracapsular cataract extraction and phacoemulsification. Extracapsular cataract extraction involves removing the lens in a relatively intact condition by use of a vectus or similar surgical instrument. Phacoemulsification involves contacting the lens with the vibrating cutting tip of an ultrasonically driven surgical handpiece to emulsify the lens, thereby allowing the emulsified lens to be aspirated from the eye. Although extracapsular cataract extraction has been the preferred surgical technique, phacoemulsification has become increasingly popular, in part because the cutting tip of the ultrasonic handpiece requires only a relatively small (approximately 3 to 3.5 millimeter) tunnel incision.

A typical posterior chamber IOL comprises an artificial lens (“optic”) and at least one support member (“haptic”) for positioning the IOL within the capsular bag. The optic may be formed from any of a number of different materials, including polymethylmethacrylate (PMMA), polycarbonate, silicon and soft acrylics, and it may be hard, relatively flexible or even fully deformable so that the IOL can be rolled or folded prior to insertion. The haptics generally are made from some resilient material, such as polypropylene, PMMA or soft acrylic. IOL's may be characterized as either “one-piece” or “multi-piece.” With one-piece IOL'S, the haptic and the optic are formed integrally as a blank and the IOL is then milled or lathed to the desired shape and configuration. Multi-piece IOL's are formed either by attaching the haptic to a preformed optic or by molding the optic around the proximal end of the haptic.

Increasingly, surgeons are making their initial incisions into the eye in the clear cornea. Clear cornea incisions have several advantages, but can be difficult to relocate once the knife is removed from the incision. Some surgeons will place a stain or dye into the eye prior to making the incision. Some of the stain or dye is pulled into the incision by the knife as the incision is being made, thereby marking the incision for the surgeon. Such as technique requires a separate step to place the stain or dye into the eye and the stain or dye flows uncontrollably over the eye, far from the incision.

Therefore, a need continues to exist for a simple method for marking the incision site in an eye during cataract surgery.

### BRIEF SUMMARY OF THE INVENTION

The present invention improves upon the prior art by providing a surgical knife containing a stain or dye coating. The stain or dye will dissolve or melt upon entry into the incision, thereby neatly marking the location of the incision.

Accordingly, one objective of the present invention is to provide a surgical knife capable of marking an incision site.

Another objective of the present invention is to provide a surgical knife containing a stain or dye coating.

These and other advantages and objectives of the present invention will become apparent from the detailed description and claims that follow.

### BRIEF DESCRIPTION OF THE DRAWINGS

FIG. 1 is a plan view of the surgical knife of the present invention.

FIG. 2 is a cross-sectional view of the knife illustrated in FIG. 1 taken along line 2-2.

### DETAILED DESCRIPTION OF THE INVENTION

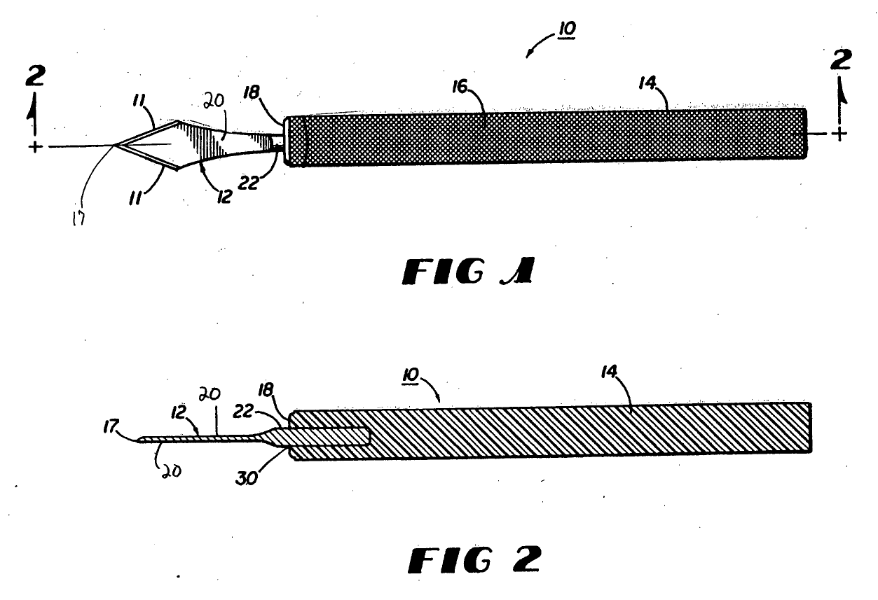

As can be seen in FIGS. 1 and 2, surgical knife 10 of the present invention includes a blade 12 and a handle 14. Knife 10 may be either reusable or disposable. If knife 10 is reusable, blade 12 may be made of any suitable material such as stainless steel or titanium and handle 14 may be made from stainless steel, titanium, or aluminum. If knife 10 is disposable, handle 14 also may be made of suitable thermoplastic, fiberglass or composite material. Handle 14 is preferably cylindrical, although other cross-sectional shapes may also be used, and may contain knurling or other suitable roughening 16 to make handle 14 more positive to grip.

As can be seen in FIG. 2, blade 12 preferably has a thin cross-section (approximately 0.5 mm) and may be formed either from a sheet material or by flattening the end of round wire 22 with an approximate diameter of 1.5 mm. Blade 12 may be straight, as shown in FIG. 2, or angled relative to handle 14. Blade 12 is attached to end 18 of handle 14 by any conventional means such as integrally molding blade 12 within handle 14 or a collet (not shown). Tip 17 is preferably between 1.5 mm and 2.5 mm wide so as to fit easily through the typical incision used for the phacoemulsification cutting tip. Cutting edges 11 are preferable ground at an angle of approximately between 25° and 55° relative to the plane in which blade 12 lays. Blade 12 preferably has is coated with stain or dye 20, such as Rose Bengal. Dye 20 can either be water-soluble, so that contact with the incision dissolves dye 20 and dye 20 can be flushed from the eye easily upon completion of the surgical procedure, or dye 20 can be temperature sensitive so that warming on dye 20 within the incision cause dye 20 to melt.

This description is given for purposes of illustration and explanation. It will be apparent to those skilled in the relevant art that changes and modifications may be made to the invention described above without departing from its scope or spirit.

I claim:

1. A surgical knife, comprising: a handle and a blade attached to the handle, the blade being coated with a stain or dye.

2. The surgical knife of claim 1 wherein the stain or dye is Rose Bengal.
